(19) United States
(12) Patent Application Publication
(10) Pub. No.: US 2022/0392570 A1
(43) Pub. Date: Dec. 8, 2022

(54) METHOD FOR SCREENING IVF EMBRYOS

(71) Applicant: GenEmbryomics Pty. Ltd., Point Cook (AU)

(72) Inventor: Nicholas Mark MURPHY, Point Cook (AU)

(73) Assignee: GenEmbryomics Pty. Ltd., Point Cook (AU)

(21) Appl. No.: 17/770,580

(22) PCT Filed: Oct. 21, 2020
(86) PCT No.: PCT/AU2020/051134

§ 371 Date: Apr. 20, 2022

(30) Foreign Application Priority Data
Oct. 22, 2019 (AU) 2019903966

(51) Int. Cl. G16B 20/20; C12Q 1/6869; G16B 20/10

(52) U.S. Cl. CPC G16B 20/20; C12Q 1/6869; G16B 20/10

(57) ABSTRACT

The present invention relates to methods of screening in vitro fertilization (IVF) embryos for pathogenic genetic variations, such as single nucleotide polymorphisms. In particular, the present invention relates to methods of screening IVF embryos using whole genome sequencing (WGS) data. The present invention also relates to methods of screening in vitro fertilization (IVF) embryos for phenotypic traits.

### RELATED APPLICATION

The present application claims priority from Australian Provisional Patent Application number 2019903966, filed 22 Oct. 2019 and entitled “Method for screening IVF embryos”. The entire contents of that earlier application are hereby incorporated by reference.

### FIELD OF THE INVENTION

The present invention relates to methods of screening in vitro fertilization (IVF) embryos for pathogenic genetic variations, such as single nucleotide polymorphisms. In particular, the present invention relates to methods of screening IVF embryos using whole genome sequencing (WGS) data.

### BACKGROUND OF THE INVENTION

There are as many as 9,000 known syndromic diseases for which genetic causality can be observed. Single mutations to one or both copies of ˜5,000 human genes can cause one of these disorders. Preconception genetic screening is increasingly utilised to determine a female or male reproductive partner's carrier-status for these damaging variations, but is generally limited to a subset of several hundred high-risk diseases. Performing genome screening on an embryo in vitro would limit transmission of genetic variations responsible for childhood-onset diseases. Between 0.5 to 5% of infants are born with a genetic condition or disease; the application of whole genome sequencing (WGS) to IVF conceived embryos provides an opportunity to screen for hereditary syndromic genetic diseases, in addition to identifying the more technically challenging de novo mutations. Approximately 30 to 100 de novo mutations are introduced in embryos, and though they carry a fraction of the absolute risk of causing disease relative to familial diseases, when they express dominance or are introduced as a compound heterozygote, they tend to produce more severe pathogenic phenotypes.

Clinical in vitro fertilisation (IVF) has included preimplantation genetic testing (PGT) for over two decades, applying available technology and methods. The aim has been to maximise the likelihood of a healthy baby by screening embryos for chromosomal aneuploidies and/or by limiting transmission of known hereditary single-gene mutations. The most recent major PGT development is to apply low-coverage next-generation sequencing (NGS), to detect chromosomal aneuploidies, structural variations (SV's) and large copy number variations (CNVs).

To date, WGS has been adopted in only a limited number of assisted reproduction cases, principally due to the high monetary price. Similarly, WGS on genomic DNA from embryos presents a significant challenge due to the low amount of starting DNA (approximately 4 to 12 copies) in a few cells biopsied from the embryo. In this regard, methods to amplify the amount of DNA from IVF embryo biopsies introduces mutations at a rate of about one in every 1,000 to 10,000 bases. Thus, WGS data from IVF embryo biopsies has a high error rate, making it difficult to accurately determine if the embryo is at risk of having a pathogenic genetic variation.

There is therefore a need to develop methods that can be used to screen IVF embryos for pathogenic genetic variations using WGS data that deal with the high error rate present in such data.

### SUMMARY OF THE INVENTION

The present inventor has identified that by combining trio testing to identify inherited variations and a variation allele frequency filter for de novo variations in WGS data, IVF embryos can be successfully screened for pathogenic genetic variations. Accordingly, in an aspect, the present invention provides a method of screening an in vitro fertilization (IVF) embryo for pathogenic genetic variations, the method comprising

a) obtaining whole genome sequencing data from the embryo, the embryo's male parent, and the embryo's female parent,

b) aligning the embryo sequencing data to a reference genome and identifying variations in the embryo sequencing data relative to the reference genome,

c) aligning the male parent's sequencing data and the female parent's sequencing data to the reference genome and identifying variations present in the parent sequencing data relative to the reference genome,

d) comparing the variations identified in step b) with those identified in step c) to identify inherited genetic variations in the embryo, wherein the inherited genetic variations are present in the embryo sequencing data and at least one parent's sequencing data,

e) filtering the variations identified in step b) that were not identified as inherited genetic variations in step d) through a variation allele frequency (VAF) threshold, wherein the filtered variations having a VAF above the threshold are identified as de novo genetic variations, and

f) comparing the inherited genetic variations and the de novo genetic variations to a database of known pathogenic genetic variations to determine if the embryo is at risk of having a pathogenic genetic variation.

As the skilled person would appreciate, the methods of the present invention address the problem of error-prone embryo-derived WGS data by using a combined approach that can identify both inherited pathogenic variations, using a comparison to the parent sequencing data (‘trio testing’), and de novo pathogenic variations, by using a suitable VAF threshold filter.

In some embodiments, the method further comprises using one or more pathogenicity prediction algorithms to predict if any of the genetic variations not identified as known pathogenic genetic variations in step f) are pathogenic genetic variations. Thus, in addition to using a database of known pathogenic genetic variations to identify variations previously classified as pathogenic (or likely pathogenic), the methods of the invention can also use prediction algorithms to assess the risk of pathogenicity associated with any of the identified variations that have not been previously classified as pathogenic in the database.

In some embodiments, the one or more pathogenicity prediction algorithms include SIFT, Polyphen2 HVAR, MutationTaster2, MutationAssessor, FATHMM, FATHMM MKL. In one embodiment, two or three or four or five or six or more pathogenicity prediction algorithms are used. When multiple pathogenicity prediction algorithms are used the proportion of those that predict a variation as being pathogenic can be used as a measure of certainty of the pathogenicity of that variation.

In some embodiments, predicting a variation to be a pathogenic genetic variation requires that a variation has a MPC score of greater than about 2 and/or a Phred scaled CADD score of greater than about 20. In some embodiments, the Phred scaled CADD score is required to be greater than 25, or greater than about 30, or greater than about 35.

In some embodiments, the VAF threshold in step e) is between 0.25 to 0.45 or 0.3 to 0.4 or is about 0.35. In some embodiments, the VAF threshold is at least 0.25, at least 0.27, at least 0.30, at least 0.32, or at least 0.35. As will appreciated by those skilled in the art, the VAF threshold can be chosen according to relative amount of risk that the embryo's parents are willing to take. For example, a younger (e.g., more fertile) couple may be inclined to choose a lower VAF threshold to ensure that no true-positive de novo pathogenic genetic variations are missed, albeit resulting in more false positive de novo pathogenic genetic variations likely being identified. Conversely, an older couple, for example with less available embryos to screen, may be inclined to choose a higher VAF threshold.

Due to the high error rate associated with WGS data from amplified embryonic DNA, there is a risk that a true-positive, low VAF, pathogenic de novo variation could be filtered out by the VAF threshold. Therefore, in some embodiments, variations having a VAF below the threshold are also examined for their potential to be pathogenic, whether or not they are “real” genetic variations. Accordingly, in some embodiments, variations having a VAF below the threshold are compared to the database of known pathogenic genetic variations to determine if the embryo is at risk of having a pathogenic genetic variation. In some embodiments, the variations having a VAF below the threshold which are not identified as known pathogenic genetic variations are further assessed by one or more pathogenicity prediction algorithms to predict if any are pathogenic genetic variations.

In some embodiments, the database of known pathogenic genetic variations is ClinVar. Other databases are also suitable. For instance, in some embodiments the database of known pathogenic genetic variations is HGMD, OMIM, or ACMG. In some embodiments, the inherited genetic variations and the de novo genetic variations are compared against 2 or 3 or more databases of known pathogenic genetic variations.

In some embodiments, determining that a genetic variation is a pathogenic genetic variation in step f) requires that the genetic variation has a clinical significance value of ‘pathogenic’ or ‘likely pathogenic’ in the ClinVar database. Such variations are considered to be higher risk and would more likely warrant deciding not to transfer the embryo, for example. Other database metrics can also be used to assess the confidence in the clinical significance of a pathogenic variation. For example, the level of confidence in the pathogenicity associated with a genetic variation in a database can be determined by how well reviewed or by the level of consensus in that annotation. For instance, in some embodiments, determining that a genetic variation is a pathogenic genetic variation in step f) requires that the variation has a review status in the ClinVar database of at least two or at least three or four stars.

In some embodiments, the genetic variations include single nucleotide polymorphisms (SNPs), insertions or deletions (indels), copy number variations (CNVs), and/or structural variations.

In some embodiments, the inherited genetic variations include autosomal dominant, autosomal recessive, compound heterozygous, and/or X-linked genetic variations.

In some embodiments, the whole genome sequencing data from the embryo is obtained by

a) culturing the embryo,

b) performing a biopsy on the embryo,

c) amplifying genomic DNA from the biopsy, and

d) sequencing the amplified genomic DNA.

Suitable methods for culturing, performing biopsies, amplifying DNA, and sequencing the DNA will be known by those skilled in the art.

In some embodiments, the biopsy is a trophectoderm biopsy. In some embodiments, the trophectoderm biopsy is performed on day 5 or day 6 of culture. Such methods are advantageous in that they maximize the copy number of genomic DNA obtained from the embryo without significantly adversely affecting the viability of the embryo.

In some embodiments, the embryo DNA is obtained from culture media instead of embryo biopsy, either ‘free’ DNA in the culture media or blastocyst culture conditioned medium combined with blastocoel fluid.

In some embodiments, the genomic DNA is amplified from the biopsy using a whole genome amplification (WGA) method. In some embodiments, the genomic DNA is amplified from the biopsy using multi-displacement amplification (MDA).

Any nucleic acid sequencing platform is suitable for performing sequencing of the genomic DNA, including high-throughput DNA sequencing methods (also commonly referred to as “next-generation sequencing” or “NGS”). Thus, in some embodiments, the amplified genomic DNA is sequenced using a high throughput sequencing method. In some embodiments, the genomic DNA is sequenced using DNA nanoball sequencing. In some embodiments, the DNA nanoball sequencing is performed with combinatorial probe anchor ligation (cPAL).

In some embodiments, the whole genome sequencing data covers at least 60%, at least 70%, at least 80%, at least 90%, or at least 95% of the embryo's genome sequence.

The whole genome sequencing data is aligned to a reference genome so that any differences between the embryo's genome sequence (and the parents) and the reference can be identified as potential genetic variations. In some embodiments, the reference genome is a human reference genome. In some embodiments, the reference genome is a Genome Reference Consortium Human Build. In some embodiments, the reference genome is Genome Reference Consortium Human Build 37 (GRCh37) or Genome Reference Consortium Human Build 38 (GRCh38) or any future build (i.e., Build 39 or later builds).

In some embodiments, two or more embryos from the same parents are screened. In some embodiments, three or four or five or six or seven or eight or nine or ten or more embryos are screened. Advantageously, when multiple embryos are screened, there is a higher likelihood of identifying an embryo that is not determined to be at risk of having a pathogenic genetic variation, which is suitable for transferring.

In some embodiments, the method further comprises transferring the embryo into the female parent's, or a surrogate's, uterus. Thus, if the embryo is screened for pathogenic genetic variations, and is determined to not be at risk of having a pathogenic genetic variation, or has a risk which is acceptably low, then the embryo may be transplanted.

In some embodiments, the embryo is a human embryo. In some embodiments, the embryo is a non-human animal embryo. Thus, in addition to human IVF, the methods described herein can be applied to other animal embryos, for example, to screen embryos for producing livestock.

In some embodiments, one or both of the parents have a pathogenic genetic variation. Thus, the methods described herein can be performed to screen and select embryos for transfer, which are determined not to have inherited that pathogenic genetic variation.

In another aspect, the present invention provides an IVF process comprising

a) fertilizing an egg from a female parent with a sperm from a male parent,

b) culturing the fertilized egg, thereby producing an embryo,

c) screening the embryo for pathogenic genetic variations using the method described herein, and

d) transferring the embryo into the female parent's, or a surrogate's, uterus.

In another aspect, the present invention provides a method of screening an in vitro fertilization (IVF) embryo for one or more phenotypic traits, the method comprising

a) obtaining whole genome sequencing data from the embryo, the embryo's male parent, and the embryo's female parent,

b) aligning the embryo sequencing data to a reference genome and identifying variations in the embryo sequencing data relative to the reference genome,

c) aligning the male parent's sequencing data and the female parent's sequencing data to the reference genome and identifying variations present in the parent sequencing data relative to the reference genome,

d) comparing the variations identified in step b) with those identified in step c) to identify inherited genetic variations in the embryo, wherein the inherited genetic variations are present in the embryo sequencing data and at least one parent's sequencing data,

e) filtering the variations identified in step b) that were not identified as inherited genetic variations in step d) through a variation allele frequency (VAF) threshold, wherein the filtered variations having a VAF above the threshold are identified as de novo genetic variations, and

f) comparing the inherited genetic variations and the de novo genetic variations to a database of genetic variations having known phenotypic traits to determine if the embryo has the one or more phenotypic traits.

Any embodiment herein shall be taken to apply mutatis mutandis to any other embodiment or aspect unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF THE ACCOMPANYING DRAWINGS

FIG. 1 shows tables of the filter sets used for identifying pathogenic genetic variations from databases of known pathogenic variations.

FIG. 2 shows tables of the filter sets used for identifying pathogenic genetic variations using pathogenicity prediction algorithms.

FIG. 3 shows tables of the filter sets used for identifying copy number variations.

FIG. 4 shows variant allele frequency (VAF) for candidate de novo variants from a typical parent in windows of VAF 0.05.

FIG. 5 shows variant allele frequency (VAF) for candidate de novo variants from a typical embryo in windows of VAF 0.05.

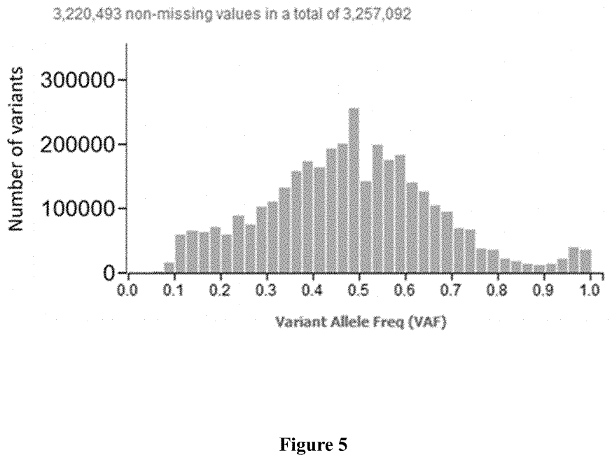

FIG. 6 shows the Quality by Depth (QD) scores for variants from a typical parent in windows of QD ˜1.06.

FIG. 7 shows the Quality by Depth (QD) scores for candidate de novo variants from a typical embryo in windows of QD ˜1.06.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., molecular genetics, bioinformatics, developmental biology, and IVF).

Unless otherwise indicated, the techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present).

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

As used herein, the term “screening” refers to a process of assessing an embryo to determine if it is at risk of having a pathogenic genetic variation. Such a process can be used to select a suitable embryo for transferring into a female's uterus, for example.

The term “pathogenic” is used herein in reference to genetic variations that are known to be or predicted to be linked to a disease. The association of one or more genetic variations with a disease can result in the disease or can represent the genetic predisposition, i.e. risk, of developing the disease.

As used herein, the terms “aligned”, “alignment”, or “aligning” refer to one or more sequences that are identified as a match in terms of the order of their nucleic acid molecules to a known sequence from a reference genome. Such alignment can be done manually or by a computer algorithm, examples including the Efficient Local Alignment of Nucleotide Data (ELAND) computer program distributed as part of the Illumina Genomics Analysis pipeline. The matching of a sequence read in aligning can be a 100% sequence match or less than 100% (non-perfect match).

The term “allele” herein refers to a sequence variant of a genetic sequence. For purposes of this application, alleles can but need not be located within a gene sequence. Alleles can be identified with respect to one or more polymorphic positions such as SNPs, while the rest of the gene sequence can remain unspecified. For example, an allele may be defined by the nucleotide present at a single SNP, or by the nucleotides present at a plurality of SNPs.

The term “sequencing” herein refers to a method for determining the nucleotide sequence of a polynucleotide, e.g. genomic DNA. Preferably, sequencing methods include as non-limiting examples next generation sequencing (NGS) methods (i.e., high throughput sequencing methods), NGS in which clonally amplified DNA templates or single DNA molecules are sequenced in a massively parallel fashion (Volkerding et al., 2009; Metzker et al., 2010).

The term “sequencing read” or “read” refers to a DNA sequence of sufficient length (e.g., at least about 30 bp) that can be used to identify a larger sequence or region, e.g. that can be aligned and specifically assigned to a chromosome or genomic region or gene.

The term “whole genome amplification” herein refers to a process whereby genomic DNA sequences present in a sample are amplified to provide multiple copies of the genome that the sequences represent.

The term “haplotype” refers to a DNA sequence comprising one or more genetic variation of interest contained on a subregion of a single chromosome of an individual. The genetic variations of a haplotype can be of the same type, e.g. all SNPs, or can be a combination of two or more types of genetic variations, e.g. combinations of SNPs and STRs. A haplotype can refer to a set of genetic variations in a single gene, an intergenic sequence, or in larger sequences include both gene and intergenic sequences, e.g., a collection of genes, or of genes and intergenic sequences. For example, a haplotype can refer to a set of genetic variations in the regulation of complement activation (RCA) locus, which includes gene sequences for complement factor H(CFH), FHR3, FHR1, FHR4, FHR2, FHR5, and F13B and intergenic sequences (i.e., intervening intergenic sequences, upstream sequences, and downstream sequences that are in linkage disequilibrium with genetic variations in the genic region). A haplotype, for instance, can be a set of maternally inherited alleles, or a set of paternally inherited alleles, at any locus.

The term “haplotyping” herein refers to a process for determining one or more haplotypes in an individual and includes use of family pedigrees, molecular techniques and/or statistical inference. Preferably, haplotypes are determined by sequencing using next generation sequencing technologies.

### In Vitro Fertilisation (IVF)

The methods described herein are used to screen IVF embryos, such that embryos which are at risk of having a pathogenic genetic variation can be identified, thereby permitting suitable embryos to be selected for transfer. IVF is a process of fertilisation where an egg is combined with sperm outside the body, i.e., in vitro. The process involves monitoring and in some instances stimulating a female's ovulatory process, removing an ovum or ova (egg or eggs) from the female's ovaries and letting sperm fertilise them in a suitable liquid in a laboratory setting. After the fertilised egg (zygote) undergoes embryonic culture for about 2-6 days, it is implanted in the same or another female's (e.g., a surrogate's) uterus, with the intention of establishing a successful pregnancy. Typically, parents will undergo IVF if they either have difficulty conceiving naturally, or are at risk of transmitting a genetic disease to the embryo.

The methods described herein are suitable for IVF embryos of any animal, provided that a suitable reference genome is available so that sequencing data can be aligned to identify potential genetic variations. For example, the embryo may be a human or other non-human animal embryo. In some embodiments, the embryo is a human embryo. In other embodiments, the embryo is a bovine, ovine, equine, porcine, canine, feline, or other non-human animal embryo.

Obtaining whole genome sequencing data for an “embryo”, as described herein, includes sequencing genomic DNA from cells from an embryo fertilized not less than about 40 hours before genotyping, cells from a blastocyst (typically an embryo at day 4, day 5 or day 6 after fertilization) as well as cells biopsied from an embryo but of extraembryonic origin, e.g., trophectoderm, or polar bodies. Thus, in some embodiments, the embryo is a two-day, three-day, four-day, five-day, six-day, or seven-day old embryo. The plural form of this term is included, such that, the term “an embryo” as used herein contemplates that more than one embryo or blastocyst may be concurrently screened or transferred according to the methods of the present invention.

It is further contemplated herein that more than one cell of an embryo may be biopsied as conditions permit. For example, one or more cells of trophectoderm may be biopsied to obtain genomic DNA for sequencing according to the methods of the present invention. The trophectoderm (also referred to as the “trophoblast”) is the outer layer cells of a blastocyst, which provide nutrients to the embryo and develop into a large part of the placenta. They are formed during the first stage of pregnancy and are the first cells to differentiate from the fertilized egg. In some embodiments, at least 2, at least 3, at least 4, at least 5, at least 6, at least 7, at least 8, at least 9, or at least 10 cells are biopsied from the embryo.

Assaying more than one cell in this way can be used to detect mosaicism in an embryo (a condition in which cells in an embryo may differ genetically from other cells in the embryo) which cannot be detected if only a single cell is biopsied. Thus, as contemplated herein, the methods of the present invention can be used to biopsy a day 5 or day 6 embryo, screen the embryo for pathogenic genetic variations, and still permit fresh transfer of the embryo on the same day. In an embodiment, the genetic sample is one, two, three to five, six to ten, or eleven to twenty cells biopsied from an embryo.

“Transferring” an IVF embryo refers to the process of placing an IVF embryo into a female subject, with the objective that the embryo will implant and result in a viable pregnancy. The female subject may be the female parent of the embryo or any other suitable female for transfer of the embryo, for example in the case of a surrogate pregnancy.

As contemplated herein, the methods of the present invention may be used to screen one or more embryos concurrently such that more than one IVF embryo deemed not at risk of having a pathogenic genetic variation may be identified and transferred. The number of such embryos that may be appropriate to transfer may be determined by one of skill in the art according to conventional methods.

### Whole Genome Sequencing

The methods described herein require obtaining whole genome sequencing data from an IVF embryo and the embryo's parents, in order to screen the embryo for pathogenic genetic variations. The term “Whole Genome Sequencing (WGS)” herein refers to a process whereby the sequence of a substantial part of the genome of an organism, for example a human, can be determined. It is not necessary that the entire genome actually be sequenced. Whole genome sequencing can be performed using any sequencing technology as described herein. Whole genome sequencing (WGS) is also referred to as full genome sequencing, complete genome sequencing, or entire genome sequencing. Reference to “whole” genome sequencing does not require that the sequencing data cover every single base in the embryo's genome. It merely requires that a sufficient portion of the genome be covered so that a prediction can be made about whether the embryo is at risk of having a pathogenic genetic variation. In some embodiments, the whole genome sequencing data covers at least 60%, at least 70%, at least 80%, or at least 90% of the embryo's genome sequence. In some embodiments, the whole genome sequencing data covers at least 60%, at least 70%, at least 80%, or at least 90% of the male parent's genome sequence. In some embodiments, the whole genome sequencing data covers at least 60%, at least 70%, at least 80%, at least 90% of the female parent's genome sequence. It is also favorable to have high sequencing depth (or “depth of coverage”) to create redundancy in the data, which enables more accurate predictions to be made about the presence or absence of a pathogenic genetic variation at a particular locus. In some embodiments, the whole genome sequencing data has average sequencing depth of at least 10×, at least 20×, at least 30×, or at least 40×.

The whole genome sequencing can be obtained by any means, including being provided by another party or by preparing suitable samples and sequencing the genomic DNA.

### Sample Preparation

Genome sequencing data can be obtained from cellular DNA, which is derived from whole cells by manually or mechanically extracting the genomic DNA from whole cells. Methods for extracting genomic DNA from whole cells are known in the art, and differ depending upon the nature of the source. In some instances, it can be advantageous to fragment the cellular genomic DNA. Fragmentation can be random, or it can be specific, as achieved, for example, using restriction endonuclease digestion. Methods for random fragmentation are well known in the art, and include, for example, limited DNAse digestion, alkali treatment and physical shearing. In other embodiments, the sample nucleic acids are obtained as cellular genomic DNA, which is subjected to fragmentation into fragments of approximately 500 or more base pairs, and to which next generation sequencing (NGS) methods can be readily applied.

To obtain the sequencing data, the method may include further preparing the genomic DNA for sequencing, for example by amplification or purification. Any suitable method may be used to prepare the genomic DNA for sequencing. In an embodiment, the genomic DNA is prepared for sequencing by amplification or universal amplification of the DNA present in the genetic sample. Furthermore, standard techniques for nucleic acid isolation and purification are known and are described in, for example, in Miller (ed.) 1972 Experiments in Molecular Genetics, Cold Spring Harbor Laboratory, Cold Spring Harbor, N.Y.; Old and Primrose, 1994 Principles of Gene Manipulation, 5th ed., University of California Press, Berkeley; Schleif and Wensink, 1982 Practical Methods in Molecular Biology; Glover (Ed.) 1985 DNA Cloning: Vols. I AND II, IRL Press, Oxford, UK; Harnes and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York City.

Nucleic acid amplification methods are also well known, including polymerase chain reaction (PCR) (PCR Protocols, A Guide to Methods and Applications, ed. Innis, Academic Press, N.Y. 1990; PCR: A Practical Approach, M. J. McPherson, et al., IRL Press (1991)); ligase chain reaction (LCR) (Landegren et al., 1988); transcription amplification (Kwoh et al., 1989); self-sustained sequence replication (Guatelli et al., 1990); Q Beta replicase amplification (Smith et al., 1997), and other RNA polymerase mediated techniques such as nucleic acid sequence based amplification, NASBA (U.S. Pat. Nos. 4,683,195 and 4,683,202); 3SR (self-sustained sequence reaction); RACE-PCR (rapid amplification of cDNA ends); PLCR (a combination of polymerase chain reaction and ligase chain reaction); SDA (strand displacement amplification); and SOE-PCR (splice overlap extension PCR).

In one embodiment, the genomic DNA is amplified from the embryo biopsy using a whole genome amplification (WGA) method. In one embodiment, the genomic DNA is amplified from the embryo biopsy using multiple displacement amplification (MDA). MDA is a non-PCR based DNA amplification technique. This method can rapidly amplify minute amounts of DNA samples to a reasonable quantity for genomic analysis. The reaction starts by annealing random hexamer primers to the template: DNA synthesis is carried out by a high fidelity enzyme, preferentially Φ29 DNA polymerase, at a constant temperature. Compared with conventional PCR amplification techniques, MDA generates larger sized products with a lower error frequency. This method has been actively used in whole genome amplification (WGA) for obtaining whole genome sequencing data. Suitable WGA methods are also described in WO08051928, for example.

It is contemplated herein that conventional methods to analyze embryonic and parental nucleic acid include methods that permit the analysis of nucleic acid from a small number of cells. Such methods may include performing a “preamplification” of DNA prior to real-time PCR using SNP locus specific primers. Such methods are a modification of methods familiar to one of skill in the art, and kits to perform such preamplification are commercially available, for example, TaqMan® PreAmp Cells-to-Ct™ Kit from Applied Biosystems. While these kits are designed to preamplify cDNA derived from RNA, they can also be used successfully on genomic DNA.

### Sequencing Library Preparation

In some embodiments, sequencing methods require the preparation of sequencing libraries. Sequencing library preparation involves the production of a random collection of adapter-modified DNA fragments, which are ready to be sequenced. Sequencing libraries of polynucleotides can be prepared from DNA or RNA, including equivalents, analogs of either DNA or cDNA, that is, complementary or copy DNA produced from an RNA template, for example by the action of reverse transcriptase. The polynucleotides may originate in double-stranded DNA (dsDNA) form (e.g. genomic DNA fragments, PCR and amplification products) or polynucleotides that may have originated in single-stranded form, as DNA or RNA, and been converted to dsDNA form. By way of example, mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, the polynucleotide molecules represent the entire genetic complement of an organism, and are genomic DNA molecules, e.g. cfDNA molecules, which include both intron and exon sequences (coding sequences), as well as non-coding regulatory sequences such as promoter and enhancer sequences. Still yet more particularly, the primary polynucleotide molecules are human genomic DNA molecules, e.g. cfDNA molecules, present in peripheral blood of a pregnant subject. Preparation of sequencing libraries for some NGS sequencing platforms require that the polynucleotides be of a specific range of fragment sizes, e.g. 0-1200 bp. Therefore, fragmentation of polynucleotides, e.g. genomic DNA, may be required. cfDNA exists as fragments of <300 base pairs. Therefore, fragmentation of cfDNA is not necessary for generating a sequencing library using cfDNA samples. Fragmentation of polynucleotide molecules by mechanical means e.g. nebulization, sonication and hydroshear, results in fragments with a heterogeneous mix of blunt and 3′- and 5′-overhanging ends. Whether polynucleotides are forcibly fragmented or naturally exist as fragments, they are converted to blunt-ended DNA having 5-phosphates and 3′-hydroxyl.

Typically, the fragment ends are end-repaired, i.e. blunt-ended, using methods or kits known in the art. The blunt-ended fragments can be phosphorylated by enzymatic treatment, for example using polynucleotide kinase. In some embodiments, a single deoxynucleotide, e.g. deoxyadenosine (A), is added to the 3′-ends of the polynucleotides, for example, by the activity of certain types of DNA polymerase such as Taq polymerase or Klenow exo minus polymerase. dA-tailed products are compatible with ‘T’ overhang present on the 3′ terminus of each duplex region of adaptors to which they are ligated in a subsequent step. dA-tailing prevents self-ligation of both of the blunt-ended polynucleotides such that there is a bias towards formation of the adaptor-ligated sequences. The dA-tailed polynucleotides are ligated to double-stranded adaptor polynucleotide sequences. The same adaptor can be used for both ends of the polynucleotide, or two sets of adaptors can be utilized. Ligation methods are known in the art and utilize ligase enzymes such as DNA ligase to covalently link the adaptor to the d-A-tailed polynucleotide. The adaptor may contain a 5′-phosphate moiety to facilitate ligation to the target 3′-OH. The dA-tailed polynucleotide contains a 5′-phosphate moiety, either residual from the shearing process, or added using an enzymatic treatment step, and has been end repaired, and optionally extended by an overhanging base or bases, to give a 3′-OH suitable for ligation. The products of the ligation reaction are purified to remove unligated adaptors, adaptors that may have ligated to one another, and to select a size range of templates for cluster generation, which can be preceded by an amplification, e.g. a PCR amplification. Purification of the ligation products can be obtained by methods including gel electrophoresis and solid-phase reversible immobilization (SPRI).

Standard protocols, e.g. protocols for sequencing, using, for example, the Illumina platform, instruct users to purify the end-repaired products prior to dA-tailing, and to purify the dA-tailing products prior to the adaptor-ligating steps of the library preparation. Purification of the end-repaired products and dA-tailed products removes enzymes, buffers, salts and the like to provide favorable reaction conditions for the subsequent enzymatic step. In one embodiment, the steps of end-repairing, dA-tailing and adaptor ligating exclude the purification steps. Thus, in one embodiment, the method of the invention encompasses preparing a sequencing library that comprises the consecutive steps of end-repairing, dA-tailing and adaptor-ligating (US 20110201507). In embodiments for preparing sequencing libraries that do not require the dA-tailing step, e.g. protocols for sequencing using Roche 454 and SOLID™ 3platforms, the steps of end-repairing and adaptor-ligating exclude the purification step of the end-repaired products prior to the adaptor-ligating.

In a next step of one embodiment of the method, an amplification reaction is prepared. The amplification step introduces to the adaptor ligated template molecules the oligonucleotide sequences required for hybridization to the flow cell. The contents of an amplification reaction are known by one skilled in the art and include appropriate substrates (such as dNTPs), enzymes (e.g. a DNA polymerase) and buffer components required for an amplification reaction. Optionally, amplification of adaptor-ligated polynucleotides can be omitted. Generally, amplification reactions require at least two amplification primers, i.e. primer oligonucleotides, which may be identical, and include an ‘adaptor-specific portion’, capable of annealing to a primer-binding sequence in the polynucleotide molecule to be amplified (or the complement thereof if the template is viewed as a single strand) during the annealing step. Once formed, the library of templates prepared according to the methods described above can be used for solid-phase nucleic acid amplification. The term ‘solid-phase amplification’ as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Solid phase PCR covers systems such as emulsions, wherein one primer is anchored to a bead and the other is in free solution, and colony formation in solid phase gel matrices wherein one primer is anchored to the surface, and one is in free solution. Following amplification, sequencing libraries can be analyzed by microfluidic capillary electrophoresis to ensure that the library is free of adaptor dimers or single stranded DNA. The library of template polynucleotide molecules is particularly suitable for use in solid phase sequencing methods. In addition to providing templates for solid-phase sequencing and solid-phase PCR, library templates provide templates for whole genome amplification.

In one embodiment, the library of adaptor-ligated polynucleotides is subjected to massively parallel sequencing, which includes techniques for sequencing millions of fragments of nucleic acids, e.g. using attachment of randomly fragmented genomic DNA to a planar, optically transparent surface and solid phase amplification to create a high density sequencing flow cell with millions of clusters. Clustered arrays can be prepared using either a process of thermocycling, as described in WO 9844151, or a process whereby the temperature is maintained as a constant, and the cycles of extension and denaturing are performed using changes of reagents. The Solexa/Illumina method referred to herein relies on the attachment of randomly fragmented genomic DNA to a planar, optically transparent surface. Attached DNA fragments are extended and bridge amplified to create an ultra-high density sequencing flow cell with millions of clusters, each containing thousands of copies of the same template (WO 0018957 and WO 9844151). The cluster templates are sequenced using a robust four-color DNA sequencing-by-synthesis technology that employs reversible terminators with removable fluorescent dyes. Alternatively, the library may be amplified on beads wherein each bead contains a forward and reverse amplification primer. The length of the sequence read is associated with the particular sequencing technology. NGS methods provide sequence reads that vary in size from tens to hundreds of base pairs. In some embodiments of the method described herein, the sequence reads are about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. It is expected that technological advances will enable single-end reads of greater than 500 bp, enabling for reads of greater than about 1000 bp when paired end reads are generated. In one embodiment, the sequence reads are 36 bp. Other sequencing methods that can be employed by the method of the invention include the single molecule sequencing methods that can sequence nucleic acids molecules >5000 bp. The massive quantity of sequence output is transferred by an analysis pipeline that transforms primary imaging output from the sequencer into strings of bases. A package of integrated algorithms performs the core primary data transformation steps: image analysis, intensity scoring, base calling and alignment.

### Alignment and Variant Calling

Once sequencing of the genomic DNA is complete, the resulting plurality of sequencing reads are mapped to determine their position in the genome, achieved by alignment. Alignment of the sequencing data is performed by comparing the sequence of a sequencing read with the sequence of a reference genome to determine the chromosomal origin of the sequenced DNA molecule.

In some embodiments, the reference genome is a human reference genome. In some embodiments, the reference genome is a Genome Reference Consortium human build. In some embodiments, the reference genome is Genome Reference Consortium Human Build 37 (GRCh37) or Genome Reference Consortium Human Build 38 (GRCh38) or a later build.

A number of computer algorithms are available for aligning sequences, including, without limitation, BLAST, BLITZ, FASTA, BOWTIE, ELAND (Illumina, Inc., San Diego, Calif., USA), Burrows-Wheeler Aligner (Li and Durbin, 2010), or GATK (DePristo et al., 2011; McKenna et al., 2010). Analysis of sequencing information for the identification of polymorphic sequences may allow for a small degree of mismatch (0-2 mismatches per sequence tag) to account for minor polymorphisms that may exist between the reference genome and the embryo or parent genomes.

In order to avoid misdiagnoses due to amplification errors such as allelic or locus drop out, it is understood herein that sequencing an allele of interest may include sequencing nucleic acid around the allele to ensure amplification accuracy. For example, the disease-causative allele may be physically linked (close together) with a non-causitive allele nearby in the DNA sequence. These two sites in the DNA are very likely to be inherited together, barring any meiotic recombination between the sites. Sites nearer each other are less likely to undergo recombination. As a result, the non-causative allele can be used as a confirmatory marker of the disease causing allele in order to avoid misdiagnosis from disease allele PCR dropout. Such techniques are familiar to one of skill in the art and include “haplotyping”. Suitable methods include those described in WO 14145820 and WO 15051006.

Once the sequencing reads have been mapped to the reference genome, the aligned sequencing data is analysed to locate variations in the sequence data relative to the reference genome, a process referred to as “variant calling”. There are myriad software packages that can automate this process, including Freebayes, SOAPsnp, realSFS, SAMtools, GATK, Beagle, IMPUTE2, MaCH, SNVmix, VarS can, DeepVariant, Somaticsniper, JointSNVMix, Avocado, NGSEP, VarDict, Reveel, or HaplotypeCaller. In some embodiments, variations in the sequencing data are identified using HaplotypeCaller by a variation quality score recalibration method.

Variant calling identifies variations present in the sequencing data that may or may not be true genetic variations present in the genome of the embryo or the embryo's parents. Accurately predicting whether these variations in the sequencing data are true genetic variations and whether those variations are pathogenic is particularly problematic for whole genome sequencing data of DNA isolated from 2 to 10 embryo cells. To obtain a sufficient amount of DNA for high throughput sequencing, the embryonic DNA is typically subjected to whole genome amplification (WGA). This process introduces errors into the DNA sequence due to the inherent error rate of the enzymes used in amplification of the DNA. In some cases, the error rate is 1 in every 1,000-10,000 bases. The present invention addresses this problem in two stages. First, the variations in the sequencing data are assessed to determine if they are likely to be real genetic variations, using trio testing (comparison to male and female parent genome sequencing data) for inherited variations and a variant allele frequency (VAF) threshold filter for de novo variations. Secondly, the variations are assessed for their likelihood of causing/contributing to disease by comparing them to a database of known pathogenic genetic variations. Any genetic variations that are present in the sequencing data but which are not classified in the database as known pathogenic (or likely pathogenic) variations can then, in some embodiments, be further assessed for potential pathogenicity using one or more pathogenicity prediction algorithms.

Genetic variations which are not identified as being inherited from either parent are filtered through a VAF threshold filter in order to identify potential de novo genetic variations. As used herein, the term “variant allele frequency” refers to the fraction of sequencing reads overlapping a genomic coordinate in the WGS data that support the variant. For example, if there are 20 sequencing reads that cover a particular SNP locus and of those 20, 10 reads have an ‘A’ in that position (wherein A is the nucleotide in the reference genome) and 10 reads have a ‘G’ (the genetic variation), then the VAF in this case will be 50%. In practice, amplification and sequencing of genomic DNA from embryo biopsies is highly error prone. Therefore, VAF filters are used to remove low VAF variations which are not likely to be real genetic variations present in the embryo genome, but are more likely due to an amplification or sequencing error. Thus, the term “filtering”, as used herein, refers to processing of input genetic variation data according to certain conditions (e.g., a VAF threshold) to produce an output. For example, the input data may include all variations not identified as inherited variations and the output may be only the variations that have a VAF above the threshold. The precise VAF threshold can be selected according to the relative risk that can be tolerated. A higher threshold is more stringent and will lead to less false positives but potentially more false negatives. Conversely, a lower threshold is less stringent and will lead to more false positives but with less false negatives.

In some embodiments, variations having a VAF below the threshold are also compared to the database of known pathogenic genetic variations to determine if the embryo is at risk of having a pathogenic genetic variation. Due to the high error rate associated with WGS data from amplified embryonic DNA, there is a risk that a true-positive, low VAF, pathogenic de novo variation could be filtered out by the VAF threshold. It is therefore advantageous in some circumstance to further assess the low VAF (i.e., below the threshold) variations for any known high risk pathogenic genetic variations (for example, by using a ‘Goal keeper’ filter). In some embodiments, the variations having a VAF below the threshold which are not identified as known pathogenic genetic variations are further assessed by one or more pathogenicity prediction algorithms to predict if any are pathogenic genetic variations. If any of the low VAF variations are identified as known pathogenic genetic variations or predicted to be pathogenic then the skilled person may manually assess whether the risk associated with that variation is sufficient to warrant not transferring the embryo or whether follow-up testing of the variation is required to determine whether it is a true-positive. The manual assessment may be performed, for example, using sequencing quality metrics, database annotation sources, gene/disease severity metrics and other available clinical information to determine if the embryo is to be screened or not. The follow-up testing could include, for example, re-biopsy and direct PCR, re-testing the whole genome amplified DNA, or testing the embryo culture media for DNA exuded by the embryo.

Candidate variations can also be assessed using other metrics, such as Quality by Depth (QD), BaseQRankSum, Strand Bias-Fisher's, Mapping Quality, MQRankSum, ReadPosRankSum, ClippingRankSum, GQ_MEAN, GQ_STDDEV, or Symmetric Odds Ratio (SOR), which can be calculated using freely available software tools in the Genome Analysis Toolkit (GATK; https://software.broadinstitute.org/gatk/). In some embodiments, identifying a genetic variation as a de novo variation includes calculating its QD score. In some embodiments, identifying a genetic variation as a de novo variation requires a QD score of at least 5, at least 6, at least 7, at least 8, at least 9, at least 10, at least 11, at least 12, at least 13, at least 14, or at least 15. In some embodiments, identifying a genetic variation as a de novo variation requires a QD score of at least 12.

The confidence level of the prediction of a variation present in the WGS data can also be assessed using a Phred scaled CADD score. A Phred scaled CADD score is a measure of the quality of the identification of individual nucleobases generated by automated DNA sequencing and is logarithmically related to the base-calling error probability for each base. It was originally developed for Phred base calling to help in the automation of DNA sequencing in the Human Genome Project. Phred scaled CADD scores are assigned to each nucleotide base call in automated sequencer traces. Phred scaled CADD scores can be used to compare the efficacy of different sequencing methods. In some embodiments, predicting a variation to be a pathogenic genetic variation requires that a variation has a Phred scaled CADD score of greater than about 20. In some embodiments, the Phred scaled CADD score is required to be greater than 25, or greater than about 30, or greater than about 35.

In another embodiment, DANN (Quang et al., 2015) is used to assess the confidence level of the prediction of a variation present in the WGS data.

### Genetic Variations

### Types of Variations

The terms “genetic variation”, “polymorphism” and “variant” are used interchangeably herein to refer to the occurrence of a variation in the genetic sequence of the embryo's genome (or the genome of either parent) relative to a reference genome. Each divergent sequence is termed an allele, and can be part of a gene or located within an intergenic or non-genic sequence. A diallelic variation has two alleles, and a triallelic variation has three alleles, and so on. Diploid organisms such as humans can contain two alleles and may be homozygous or heterozygous for allelic forms. The first identified allelic form is arbitrarily designated the reference form or allele; other allelic forms are designated as alternative or variant alleles. The most frequently occurring allelic form in a selected population is typically referred to as the wild-type form. Genetic variations encompass sequence differences that include single nucleotide polymorphisms (SNPs), tandem SNPs, small-scale multi-base deletions or insertions, called “indels” (also called deletion insertion polymorphisms or DIPs), Multi-Nucleotide Polymorphisms (MNPs), Short Tandem Repeats (STRs), restriction fragment length polymorphism (RFLP), deletions, including microdeletions, insertions, including microinsertions, duplications, inversions, translocations, multiplications, complex multi-site variants, copy number variations (CNV), and other structural variations comprising any other change of sequence in a chromosome. Differences in genomic sequences include combinations of different types of variations. For example, genetic variations can encompass the combination of one or more SNPs and one or more STR.

The term “single nucleotide polymorphism (SNP)” refers to a single base (nucleotide) polymorphism in a DNA sequence among individuals in a population. A SNP may be present within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed “synonymous” (sometimes called a silent mutation)—if a different polypeptide sequence is produced they are “nonsynonymous”. A nonsynonymous change may either be missense or “nonsense”, where a missense change results in a different amino acid, while a nonsense change results in a premature stop codon. SNPs can also arise from a deletion of a nucleotide or an insertion of a nucleotide relative to a reference allele. Single nucleotide polymorphisms (SNPs) are positions at which two alternative bases occur at appreciable frequency (>1%) in the human population, and are the most common type of human genetic variation.

An “indel” is an insertion or deletion of bases in the genome of an organism. It is typically classified among small genetic variations, measuring from 1 to 10 000 base pairs in length. A “microindel” is typically defined as an indel that results in a net change of 1 to 50 nucleotides. In coding regions of the genome, unless the length of an indel is a multiple of 3, it will produce a frameshift mutation. For example, a common microindel which results in a frameshift causes Bloom syndrome in Jewish or Japanese populations. Indels can be contrasted with a point mutation (SNP). An indel inserts and deletes nucleotides from a sequence, while a point mutation (SNP) is a form of substitution that replaces one of the nucleotides without changing the overall number of nucleotides in the DNA. An indel change of a single base pair in the coding part of an mRNA results in a frameshift during mRNA translation that could lead to an inappropriate (premature) stop codon in a different frame. Indels that are not multiples of 3 are particularly uncommon in coding regions but relatively common in non-coding regions. Indels are likely to represent between 16% and 25% of all sequence polymorphisms in humans.

As used herein, the term “structural variation” is used to refer to any variation in structure of an organism's chromosome. It comprises many kinds of variation in the genome, and usually includes microscopic and submicroscopic types, deletions, duplications, copy-number variants, insertions, inversions and translocations. Typically, structural variations affect a larger portion of sequence than SNPs but smaller than a chromosome abnormality (though the definitions have some overlap). In some embodiments, structural variants are genetic events that affect a region of greater than 50 bases. The definition of structural variation does not imply anything about frequency or phenotypical effects. Many structural variants are associated with genetic diseases, however many are not. Recent research about SVs indicates that SVs are more difficult to detect than SNPs. Approximately 13% of the human genome is defined as structurally variant in the normal population, and there are at least 240 genes that exist as homozygous deletion polymorphisms in human populations, suggesting these genes are dispensable in humans. Thus, structural variations can comprise millions of nucleotides of heterogeneity within every genome, and are likely to make an important contribution to human diversity and disease susceptibility.

The term “copy number variation” herein refers to a type of structural variation that is a variation in the number of copies of a nucleic acid sequence that is typically about 1 kb or larger present in a test sample in comparison with the copy number of the nucleic acid sequence present in a qualified sample. A “copy number variant” refers to the about 1 kb or larger sequence of nucleic acid in which copy-number differences are found by comparison of a sequence of interest in test sample with that present in a qualified sample. Copy number variants/variations include deletions, including microdeletions, insertions, including microinsertions, duplications, multiplications, inversions, translocations and complex multi-site variants. CNV encompass chromosomal aneuploidies and partial aneuplodies.

The term “short tandem repeat” or “STR” herein refers to a class of polymorphisms that occurs when a pattern of two or more nucleotides are repeated and the repeated sequences are directly adjacent to each other. The pattern can range in length from 2 to 10 base pairs (bp) (for example (CATG)n in a genomic region) and is typically in the non-coding intron region. By examining several STR loci and counting how many repeats of a specific STR sequence there are at a given locus, it is possible to create a unique genetic profile of an individual.

Once candidate genetic variations have been identified in the whole genome sequencing data, they are assessed for their likelihood of contributing to a disease. Thus, the genetic variations are assessed to determine if they are pathogenic genetic variations. A “pathogenic genetic variation”, as referred to herein, is a genetic variation that is associated with a particular disease. A pathogenic variation in a subject's genome, in this context, is not necessarily one whose absence or presence dictates whether the subject is healthy or diseased. By “pathogenic” it is merely meant that the genetic variation is thought to contribute in some manner to a disease. Thus, a single copy of a disease-causing variant for a recessive disease is considered to be pathogenic, even though the single copy alone will not cause disease. Similarly, pathogenic genetic variations include both high and low penetrance genetic variations.

### Databases of Known Pathogenic Genetic Variations

Pathogenic genetic variations can be identified, for example, by querying a database of known genetic variations which is annotated with their level of pathogenicity. Alternatively, for a genetic variation which is either not in such a database, or for which its level of pathogenicity is uncertain, pathogenicity prediction algorithms can be used to determine whether that variation is likely to be pathogenic.

Suitable databases of known pathogenic genetic variations include ClinVar (https://www.ncbi.nlm.nih.gov/clinvar/), CLINVITAE (http://clinvitae.invitae.com/), Leiden Open Variant Database (LOVD; http://www.lovd.nl/), Human Genetic Variation Database (HGVD; http://www.hgvd.genome.med.kyoto-u.ac.jp/), Online Mendelian Inheritance in Man (OMIM; http://www.omim.org/), EGL's Variant Classification Catalog (EmVClass; http://www.egl-eurofins.com/emyclass/emyclass.php), ARUP mutation database (http://www.arup.utah.edu/database/), or Carver allele-specific variation database (https://www.carverlab.org/database).

In one embodiment, the ClinVar database is used to identify known pathogenic genetic variations. ClinVar is a freely accessible, public archive of reports of the relationships among human genetic variations and phenotypes, with supporting evidence. ClinVar is therefore a record of the relationships asserted between human variation and observed health status, and the history of that interpretation. ClinVar processes submissions reporting variants found in patient samples, assertions made regarding their clinical significance (i.e., level of pathogenicity), information about the submitter, and other supporting data. The alleles described in submissions are mapped to reference sequences, and reported according to HGVS standard.

ClinVar database entries generally have one of five levels of clinical significance (pathogenicity): benign, likely benign, uncertain significance, likely pathogenic, or pathogenic according to American College of Medical Genetics and Genomics (ACMG) guidelines (Richards et al., 2015). In some embodiments, determining that a genetic variation is a pathogenic genetic variation in step f) requires that the genetic variation has a clinical significance value of pathogenic or likely pathogenic in the ClinVar database.

The level of confidence in the accuracy of variation calls and assertions of clinical significance depends in large part on the supporting evidence. Because the availability of supporting evidence may vary, particularly in regard to retrospective data aggregated from published literature, ClinVar aggregates related information from multiple groups, to reflect transparently both consensus and conflicting assertions of clinical significance. A review status is also assigned to any assertion, to support communication about the trustworthiness of any assertion. The “review status” is a four-star measure of the level of certainty associated with the asserted clinical significance of a genetic variation. For example, a database entry having a review status of four stars is considered a “practice guideline” as there is a high degree of consensus in the level of clinical significance of the variation. Conversely a review status of one star reflects a low level of consensus in the clinical significance of the variant, for example if multiple submitters have provided evidence of clinical significance but there are conflicting interpretations. Thus, in some embodiments, determining that a genetic variation is a pathogenic genetic variation in step f) of the method of the invention requires that the variation has a review status in the ClinVar database of at least two or at least three or four stars. Further information regarding ClinVar review status is available at https://www.ncbi.nlm.nih.gov/clinvar/docs/review_guidelines/.

Other factors which can be used to weigh the context of the information related to a known pathogenic variant include but are not limited to its penetrance, the statistical power of the studies underlying the information, the number and type of controls involved the studies underlying the information, whether therapeutics are known to act predictably based upon the information, whether multiple mutations in a pathway are known to cause predictable phenotypes, whether there is contradictory evidence in the knowledge base and the volume/credibility of such evidence, whether the variant or variants disrupting the same gene/pathway are frequently observed in healthy individuals, whether or not the position or region in which the variant occurs is highly evolutionarily conserved, and/or whether phenocopies which are related to the variant exist and act predictably.

### Pathogenicity Prediction Algorithms

For genetic variations that are not known to be pathogenic (i.e., are not present in a database of known pathogenic variations or are present but with low review status) one may calculate the likelihood of that genetic variation being pathogenic by using one or more pathogenicity prediction algorithms. Any suitable algorithm that is configured to assess the likelihood of a genetic variation contributing to disease can be used.

These algorithms can, for example, predict whether a genetic variant is predicted to be innocuous (e.g., using a functional prediction algorithm such as SIFT or Polyphen). The following algorithms can be used alone or in combination as a part of the pathogenicity prediction algorithm: SIFT, PolyPhen, PolyPhen2, PANTHER, SNPs3D, FastSNP, SNAP, LS-SNP, PMUT, PupaSuite, SNPeffect, SNPeffectV2.0, F-SNP, MAPP, PhD-SNP, MutDB, SNP Function Portal, PolyDoms, SNP@Promoter, Auto-Mute, MutPred, SNP@Ethnos, nsSNPanalyzer, SNP@Domain, StSNP, MtSNPscore, MutationTaster2, MutationAssessor, FATHMM, or Genome Variation Server. These algorithms and other suitable algorithms known in the art that attempt to predict the effect a variation has on protein function, activity, or regulation may be utilized. For example, predicted transcription factor binding sites, ncRNAs, miRNA targets, enhancers and UTRs can be incorporated into algorithms to carry out the data analysis. Variants associated with coding vs. non-coding regions can be treated differently. Similarly, variants associated with exons vs. introns can be treated differently. Further, synonymous vs. non-synonymous variants in a coding region can be treated differently. In some cases, the translational machinery of the subject can be considered when analysing codon changes.

In some embodiments the pathogenicity prediction algorithm determines whether a sequence associated with a variant is evolutionarily conserved. Variants occurring in those sequences which have been highly conserved evolutionarily may be expected to be more deleterious, and accordingly in some embodiments the pathogenicity prediction algorithm can keep (or remove) these, depending on the application. One measure that can be used to quantify the degree of nucleotide-level evolutionary conservation is Genomic Evolutionary Rate Profiling (GERP).

In some embodiments the pathogenicity prediction algorithm assesses the nature of the amino-acid replacement associated with a variant. For example, a Grantham matrix score can be calculated. Similarly, in some embodiments, variations are assessed according to Polymorphism Phenotyping (e.g., PolyPhen2) and/or Sorting Intolerant from Tolerant (SIFT) algorithms.

In some embodiments, the one or more pathogenicity prediction algorithms include SIFT, Polyphen2 HVAR, MutationTaster2, MutationAssessor, FATHMM, or FATHMM MKL. In some embodiments, two or three or four or five or all six algorithms are used. Thus, the number of algorithms that report a variation as being predicted to be pathogenic can be used as a level of confidence in the prediction.

Suitable methods and algorithms for predicting the pathogenicity of genetic variations include those described in WO 2015061422, US 20120310863, US 20140359422 and US 20160371431, for example.

In some embodiments, in order to predict whether a genetic variation is pathogenic, an MPC score is generated. An “MPC score” as used herein, refers to “Missense badness, PolyPhen-2, and Constraint score” as described in Samocha et al., 2017 (BioRxiv, http://dx.doi.org/10.1101/148353). MPC scores measure the increased deleteriousness of amino acid substitutions when they occur in missense-constrained regions. It can be used to combine information from orthogonal deleteriousness metrics into one score. In some embodiments, determining that a genetic variation is pathogenic requires that the genetic variation has an MPC score of greater than or equal to 2.

### Diseases Caused by Pathogenic Genetic Variations

The diseases associated with the pathogenic genetic variations that can be identified by the present methods are genetic diseases, which are illnesses caused at least in part by abnormalities in genes or chromosomes. Such diseases include monogenic, i.e. single gene diseases and polygenic, i.e. complex diseases. Single gene diseases include autosomal dominant, autosomal recessive, X-linked dominant, X-linked recessive, and Y-linked. The methods of the present invention can be used to screen embryos of parents who are carriers of alleles of such diseases, so that suitable embryos can be selected for transfer.

The present method can be used to screen embryos which are at risk of developing an autosomal dominant disease. In autosomal dominant diseases, only one mutated copy of the gene will be necessary for a person to be affected by the disease. Typically, an affected subject has one affected parent, and there is a 50% chance that the offspring will inherit the mutated gene. Conditions that are autosomal dominant sometimes have reduced penetrance, which means that although only one mutated copy is needed, not all individuals who inherit that mutation go on to develop the disease. Examples of autosomal dominant diseases include, without limitation, familial hypercholesterolemia, hereditary spherocytosis, Marfan syndrome, neurofibromatosis type 1, hereditary nonpolyposis colorectal cancer, and hereditary multiple exostoses, and Huntington's disease.

The present method can also be used to screen embryos which are at risk of developing an autosomal recessive disease. In autosomal recessive diseases, two copies of the gene must be mutated for a subject to be affected by an autosomal recessive disease. An affected subject usually has unaffected parents who each carry a single copy of the mutated gene (and are referred to as carriers). Two unaffected people who each carry one copy of the mutated gene have a 25% chance with each pregnancy of having a child affected by the disease. Examples of this type of disease include cystic fibrosis, sickle-cell disease, Tay-Sachs disease, Niemann-Pick disease, spinal muscular atrophy, Roberts syndrome, Mucopolysaccharidoses, Glycogen storage diseases, and Galactosemia. Certain other phenotypes, such as wet versus dry earwax, are also determined in an autosomal recessive fashion.

The present method can also be used to screen embryos which are at risk of developing an X-linked dominant disease. X-linked dominant diseases are caused by mutations in genes on the X chromosome. Only a few diseases have this inheritance pattern, with a prime example being X-linked hypophosphatemic rickets. Males and females are both affected in these diseases, with males typically being more severely affected than females. Some X-linked dominant conditions such as Rett syndrome, incontinentia pigmenti type 2 and Aicardi syndrome are usually fatal in males, and are therefore predominantly seen in females. Exceptions to this finding are extremely rare cases in which boys with Klinefelter syndrome (47,XXY) also inherit an X-linked dominant condition and exhibit symptoms more similar to those of a female in terms of disease severity. The chance of passing on an X-linked dominant disease differs between men and women. The sons of a man with an X-linked dominant disease will all be unaffected (since they receive their father's Y chromosome), and his daughters will all inherit the condition. A woman with an X-linked dominant disease has a 50% chance of having an affected fetus with each pregnancy, although it should be noted that in cases such as incontinentia pigmenti only female offspring are generally viable. In addition, although these conditions do not alter fertility per se, individuals with Rett syndrome or Aicardi syndrome rarely reproduce.

The present method can also be used to screen embryos which are at risk of developing an X-linked recessive disease. X-linked recessive conditions are also caused by mutations in genes on the X chromosome. Males are more frequently affected than females, and the chance of passing on the disease differs between men and women. The sons of a man with an X-linked recessive disease will not be affected, and his daughters will carry one copy of the mutated gene. A woman who is a carrier of an X-linked recessive disease (XRXr) has a 50% chance of having sons who are affected and a 50% chance of having daughters who carry one copy of the mutated gene and are therefore carriers. X-linked recessive conditions include without limitation, the serious diseases Hemophilia A, Duchenne muscular dystrophy, and Lesch-Nyhan syndrome as well as common and less serious conditions such as male pattern baldness and red-green colour blindness. X-linked recessive conditions can sometimes manifest in females due to skewed X-inactivation or monosomy X (Turner syndrome).

The present method can also be used to screen embryos which are at risk of developing a Y-linked disease. Y-linked diseases are caused by mutations on the Y chromosome. Because males inherit a Y chromosome from their fathers, every son of an affected father will be affected. Because females inherit an X chromosome from their fathers, female offspring of affected fathers are never affected. Since the Y chromosome is relatively small and contains very few genes, there are relatively few Y-linked diseases. Often the symptoms include infertility, which may be circumvented with the help of some fertility treatments. Examples are male infertility and hypertrichosis pinnae.

The present method can also be used to screen embryos which are at risk of developing genetic diseases that are complex, multifactorial, or polygenic, meaning that they are likely associated with the effects of multiple genes in combination with lifestyle and environmental factors. Multifactorial diseases include for example, heart disease and diabetes. Although complex diseases often cluster in families, they do not have a clear-cut pattern of inheritance. On a pedigree, polygenic diseases do tend to “run in families”, but the inheritance does is not simple as is with Mendelian diseases. Strong environmental components are associated with many complex diseases e.g., blood pressure. The present method can be used to identify pathogenic genetic variations that are associated with polygenic diseases including but not limited to asthma, autoimmune diseases such as multiple sclerosis, cancers, ciliopathies, cleft palate, diabetes, heart disease, hypertension, inflammatory bowel disease, mental retardation, mood disease, obesity, refractive error, and infertility.

### Computer Implemented Methods

It is envisaged that the methods of the present disclosure, at least in part, may be implemented by a system such as a computer. For example, the system may be a computer system comprising one or a plurality of processors which may operate together (referred to for convenience as “processor”) connected to a memory. The memory may be a non-transitory computer readable medium, such as a hard drive, a solid state disk or other medium. Software, that is, executable instructions or program code, such as program code grouped into code modules, may be stored on the memory, and may, when executed by the processor, cause the computer system to perform functions such as determining that a task is to be performed to assist a user to; receive whole genome sequencing data; process the data, e.g., aligning the data to a reference genome to identify variations; compare variations between the embryo and parents to identify inherited genetic variations; apply various filters to the identified variations, e.g., a VAF threshold filter; compare the variations to a database of known pathogenic variations; and/or output any pathogenic genetic variations that have been identified in the embryo's genome.

In another embodiment, the system may be coupled to a user interface to enable the system to receive information from a user and/or to output or display information. For example, the user interface may comprise a graphical user interface, a voice user interface or a touchscreen.

In an embodiment, the system may be configured to communicate with at least one remote device or server across a communications network such as a wireless communications network. For example, the system may be configured to receive information from the device or server across the communications network and to transmit information to the same or a different device or server across the communications network. In other embodiments, the system may be isolated from direct user interaction.

### EXAMPLES

### Materials and Methods

### Study Participants

Couples who had undergone PGT for single gene diseases provided further informed consent to having WGS on both themselves and their biopsied embryos selected for the study (Handyside et al., 2010). Each male and female partner who participated were given the option to have the results of their DNA and biopsied embryo samples either reported or withheld. The study and protocol was approved following accepted recommendations by the Monash Health Human Research Ethics Committee. All subjects gave written informed consent in accordance with the Declaration of Helsinki.

### Library Preparation and Sequencing

Stored genomic DNA (gDNA) samples from five couples that had been used as reference templates for Karyomapping PGT cases were selected for WGS at BGI Genomics (Tai Po, Hong Kong). The DNA had been extracted from whole blood using a ReliaPrep™ Blood gDNA Miniprep System (Promega, USA). For the isolation of embryonic DNA, ICSI-created embryos belonging to the five PGT cases underwent trophectoderm (TE) biopsy on day +5 or +6 of culture, using laser or mechanical techniques, to remove an estimated 4-10 TE cells. Biopsied cells were washed three times in a solution of 1×PBS buffer (Cell Signalling Technologies, USA) and 1× polyvinylpyrrolidone (Cook Medical, Australia) followed by whole genome amplification (WGA) by multi-displacement amplification with SureMDA system (Illumina, USA) as per manufacturer's instructions. Samples for WGS were selected based on Karyomapping quality control metrics which indicated a SNP call-rate on the HumanCytoSnp-12 BeadArray of >96% and allele dropout and miscall rates of <1%. Between 1-2 ug of genomic DNA and WGA product were sent to BGI Genomics (Tai Po, Hong Kong) for sequencing with the BGI-SEQ500. Briefly, the DNA samples were fragmented to approximately 350 bp with a E220 Covaris (Covaris Inc.) followed by 3′ end-repair, adaptor ligation and amplification by ligation-mediated PCR, single strand separation and cyclization. DNA Nanoballs were produced with rolling-circle amplification, placed in patterned nanoarrays which are pair-end read by the BGI-SEQ500 (Patch et al., 2018).

### Read Processing

Read-processing through to Variation Call Format (VCF) was performed in accordance with Genome Analysis Toolkit (GATK) through BGI Online (Van der Auwera et al., 2013). Samples were mapped to the human reference genome (GRCh37/HG19) with Burrows-Wheeler Aligner (Li and Durbin, 2010), PCR duplicates removed using Picard tools (https://broadinstitute.github.io/picard/), and local realignment with GATK (DePristo et al., 2011; McKenna et al., 2010) and variations called with HaplotypeCaller by the variation quality score recalibration method.

### SNP and Indel Analysis

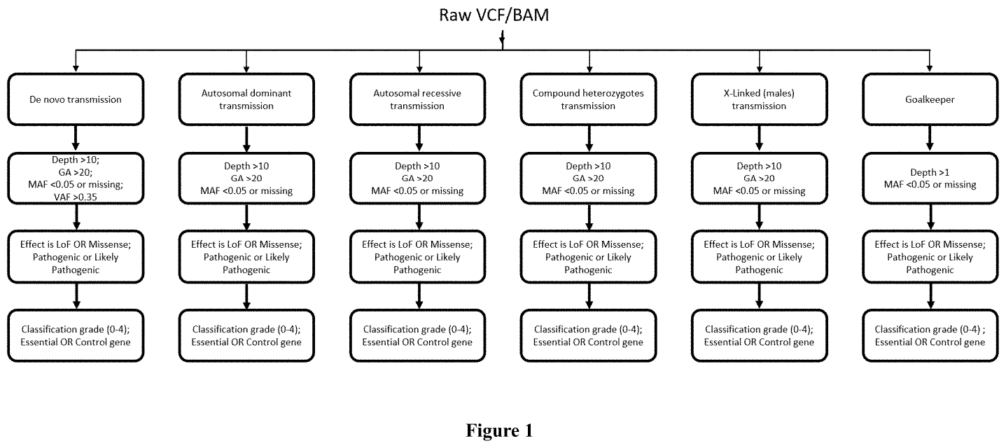

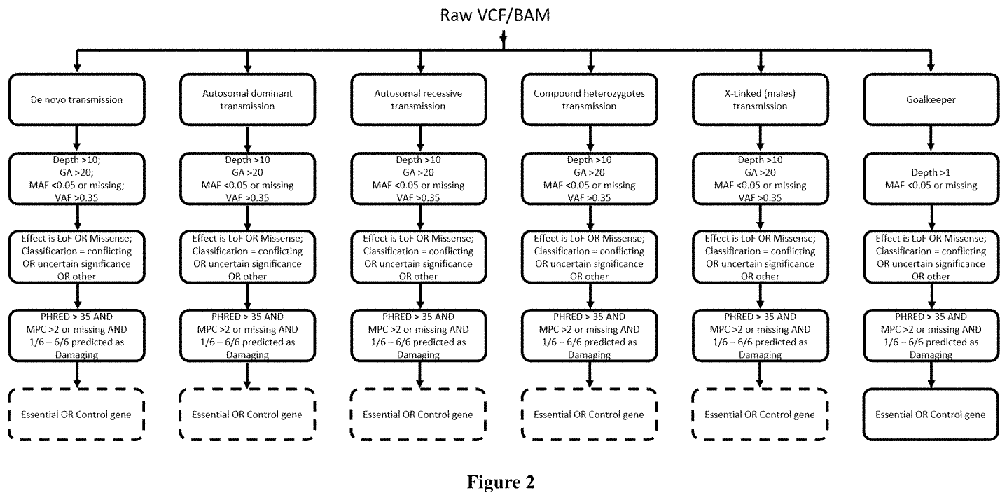

Analysis was guided by the ACMG Standards and Guidelines for interpretation of sequence variations (Rehm et al., 2013; Richards et al., 2015). Each partner and embryo BAM and raw VCF files were imported into VarSeq (GoldenHelix, USA). Variation filtering workflows were arranged for the inheritance modes of: i. dominant heterozygous, ii. recessive homozygous, iii. compound heterozygous, iv. X-linked, v. de novo and vi. a ‘Goalkeeper’ filter set to capture variations within regions of potential allelic dropout or low variant allele frequency (FIG. 1). For unclassified variations (i.e., variations that were not found in a database of known pathogenic genetic variations), a stringent pathogenicity functional prediction filter was enabled for six prediction algorithms; SIFT, Polyphen2 HVAR, MutationTaster2, MutationAssessor, FATHMM and FATHMM MKL (Ng and Henikoff, 2003; Adzhubei et al., 2013; Schwarz et al., 2014; Reva et al., 2011; Shihab et al., 2014). If any one of the six algorithms predicted a variation as ‘damaging’, the variation was retained for continued assessment in the filter chain. MPC scores of >2 and/or a final PHRED score of >35 concluded the mutation prediction filter set (FIG. 2) (Samocha et al., 2017; Kircher et al., 2014). Tandem repeats were calculated with the Expansionhunter tool to determine tandem repeat numbers on both embryos and male and female reproductive partners (Dolzhenko et al., 2017). Calculation was performed at BGI Genomics for the following loci: Cbl proto-oncogene (CBL), Atrophin 1 (ATN1), Ataxin 2 (ATXN2), Ataxin 3 (ATXN3), Junctophilin 3 (JPH3), Calcium channel, voltage-dependent, P/Q type, alpha 1A subunit (CACNA1A), Dystrophia myotonica-protein kinase (DMPK), Cystatin B (CSTB), Ataxin 10 (ATXN10), Ataxin 7 (ATXN7), Huntingtin (HTT), Protein phosphatase 2, regulatory subunit B, beta (PPP2R2B), Ataxin 10 (ATXN1), Chromosome 9 open reading frame 72 (C9ORF72), Frataxin (FXN), Androgen receptor (AR) and Fragile X mental retardation 1 (FMR1) on all adult and embryo samples.

### Copy Number and Structural Variation

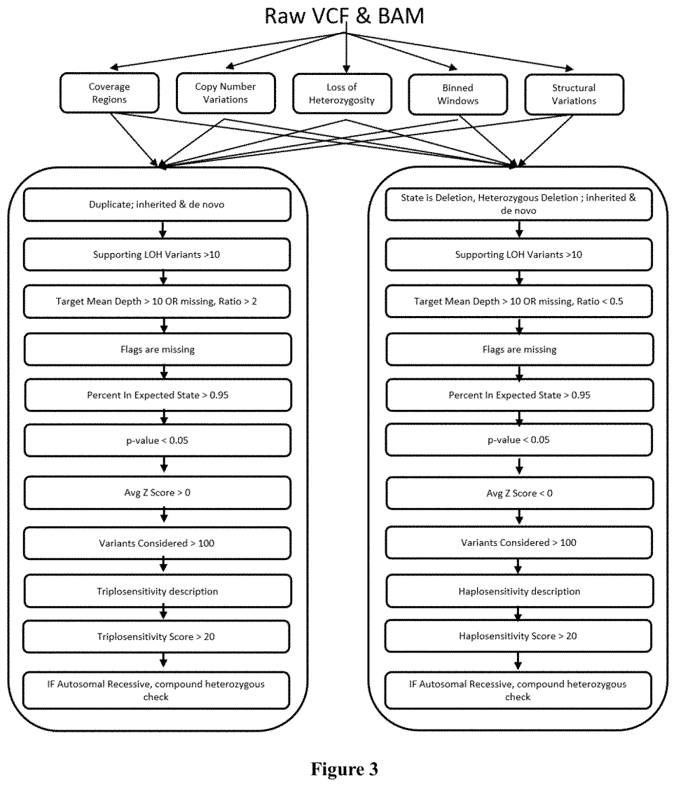

CNV calls were made using CNVnator (v.0.2.7) (Abyzov et al., 2011) and structural variations with Breakdancer (Fan et al., 2014) and CREST (Wang et al., 2011). A second analysis was performed by binning into 10 kb windows and annotating using ClinGen Gene Dosage Sensitivity (27 Sep. 2017) filtered through the calling LoH in >95% (Kearney et al., 2011; Riggs et al., 2018). CNVnator and Breakdancer calls were imported into Varseq compared to inherited CNVs from each parent, and categorised as having dosage pathogenicity for either haploinsufficiency or triplosensitivity. Loss of Heterozygosity (LoH) regions (>100 and 95% of variations) were trio-called compared to couple's LoH regions. Filtering was applied for the haploinsufficiency and triplosensitivity categories of ‘Sufficient evidence for dosage pathogenicity’ or ‘Gene associated with autosomal recessive phenotype’ and called for pathogenicity using the Target Copy Number State for Proband per sample by applying a ratio of >2.0 with Z-score >0 for duplications and <0.5 with Z-score <0, an average targeted mean depth >5 and lacking QC Flags (high control variation, low control depth, low Z Score, within regional IQR). CNVs with recessive inheritance were cross-checked against the autosomal recessive SNP and Indel variations (FIG. 3).

### Example 1—PGT Variation Validation

All participating couples consented to WGS and elected to receive results for themselves and PGT embryos. Three families had undergone PGT for autosomal dominant conditions, one for an autosomal recessive and one X-Linked (Table 1). For three of the five families, at least one euploid embryo was available from each of the possible carrier status scenarios (i.e. affected/carrier/unaffected). Heterozygote variations called from VCF files were compared to those previously obtained through Karyomapping, showing >99% concordance with WGS calls.

TABLE 1
Previously known variations in parents
from PGT and number of affected embryos

| | | | Embryo | |
|---|---|---|---|---|
| PGT | PGT Gene; Disease | | status | |
| Couple | (n = 10) | Inheritance | (n = 11) | Variation |
| A | PTPN11; Noonan | Autosomal | 1x Affected | SNP |
| | Syndrome 1 | Dominant | | |
| B | GLA; Fabry Disease | X-Linked | 1x Affected | SNP |
| | | recessive | | |
| C | BRCA2; Multiple | Autosomal | 1x Affected, | Indel |
| | neoplasms | Dominant | 1x | |
| | | | Unaffected | |
| D | CFTR; | Autosomal | 1x Affected | SNP |
| | Cystic Fibrosis | Recessive | 1x Carrier | |
| | | | 1x | |
| | | | Unaffected | |
| E | KRT10; | Autosomal | 1x Affected | SNP |
| | Epidermolytic | Dominant | 3x | |
| | Hyperkeratosis | | Unaffected | |

### Example 2—Sequencing and Mapping of Amplified Embryo DNA to Partner Genomic DNA

Sequencing depth was comparable between the amplified trophectoderm-biopsy DNA from embryos and the couple genomes (average of 48.2× versus 46.1×). Embryos were equivalent to parent genomic DNA samples for raw and clean reads, bases aligned and ratio of transitions to transversion mutations (Tv/Ti; 2.071 versus 2.081, Table 2).

TABLE 2
Sequencing and variation statistics

| | Couple | SD. | Embryo | SD. |
|---|---|---|---|---|
| Total SNPs | 3434300.5 | 2654.5 | 3581783.3 | 20036.6 |
| Fraction of SNPs | 99.1 | 0.0 | 97.5 | 0.6 |
| in dbSNP (%) | | | | |
| Fraction of SNPs | 97.6 | 0.2 | 94.2 | 0.7 |
| in 1000genomes | | | | |
| (%) | | | | |
| Novel | 22487.5 | 710.5 | 78034.3 | 20426.6 |
| Homozygous | 1400593.0 | 34778.0 | 1425083.0 | 6921.2 |
| Heterozygous | 2033707.5 | 32123.5 | 2156700.3 | 24601.5 |
| Intronic | 1353220.5 | 2800.5 | 1395884.3 | 9904.3 |
| 5′ UTRs | 4122.5 | 45.5 | 4416.8 | 49.6 |
| 3′ UTRs | 22034.5 | 95.5 | 22576.5 | 138.9 |
| Upstream | 46326.0 | 141.0 | 48901.5 | 228.7 |
| Downstream | 45418.5 | 146.5 | 47617.5 | 311.2 |
| Intergenic | 1934455.0 | 717.0 | 2032045.8 | 10056.2 |
| Ti/Tv | 2.1 | 0.0 | 2.1 | 0.0 |
| Total InDels | 859161.0 | 17947.0 | 860210.3 | 17020.4 |
| Fraction of | 75.8 | 0.6 | 74.9 | 0.4 |
| InDels in dbSNP | | | | |
| (%) | | | | |
| Fraction of | 53.6 | 0.6 | 52.6 | 0.5 |
| InDels in | | | | |
| 1000genomes | | | | |
| (%) | | | | |
| Novel | 190574.5 | 8988.5 | 198125.3 | 7305.3 |
| Homozygous | 285024.5 | 5710.5 | 284975.5 | 2923.9 |
| Heterozygous | 574136.5 | 23657.5 | 575234.8 | 19507.1 |
| Intronic | 363761.0 | 6904.0 | 364131.5 | 6674.4 |
| 5′ UTRs | 745.0 | 30.0 | 805.8 | 14.7 |
| 3′ UTRs | 6383.0 | 117.0 | 6485.3 | 87.0 |
| Upstream | 14015.0 | 315.0 | 14271.8 | 131.0 |
| Downstream | 13942.0 | 302.0 | 14051.0 | 150.9 |
| Intergenic | 458209.5 | 10244.5 | 458179.0 | 9990.6 |

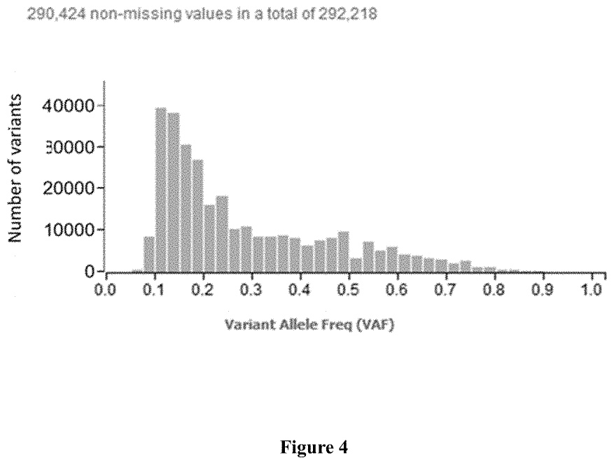

Genome coverage for embryos and couples was comparable at 4× and 10×. However, at 20× coverage was decreased for biopsied embryos to 87.5% compared to 96.4% from parent DNA. Assembly and mapping metrics for the SNP/Indel calls were highly concordant between embryos and couples, with the exception of novel SNPs that were called an average 85,527 (S.D. 29,576.6) for embryos, compared to couples at having an average of 21,663 novel SNPs (S.D. 1102.4). Accordingly, variation filters were arranged to remove false positive de novo variations, while retaining any deemed clinically actionable. For male and female parent sequencing data, a variation allele frequency (VAF) peak was observed at 0.5, as expected for true heterozygous variations. Another, false positive, peak was present at about 0.25 (ranged from 0.08-0.34, FIG. 4). Accordingly, de novo variations called in the embryo sequencing data with VAF <0.35 were filtered out to reduce the risk of false positives, but high risk de novo variations that were filtered out were retained for manual examination and potential curation (using a ‘Goal keeper’ filter). Interestingly, SNPs involved in a deletion >1 bp were observed to have a higher VAF than those involving a base change though we did not alter the filtering based on this as the upper limit was approximately consistent.

### Example 3—Variation Trio-Calling

Variations that were previously identified in PGT were confirmed in both the male/female partners and embryos with complete concordance in all 11 embryos (Table 1). One embryo's PGT variation had a substantially lower variation allele frequency (VAF=0.143; 3/21 reads), though the presence of the mutation was confirmed by showing concordance to the mutation in the parent which were typed by genetic pathology labs for the couples to undergo PGT by sequencing. For the embryos, linkage disequilibrium to common SNPs was used to known mutation carriers, affected and unaffected parents and/or siblings. After eliminating variations which were PGT indications, an average of 0.82 ‘classified’ and 1.27 ‘unclassified’ recessive de novo variations were called per embryo, compared to 1.27 classified and 0.45 unclassified de novo dominant variations that ranged between 1 and 2 stars (out of 4) for ClinVar review status. The mean number of regions called with Loss of Heterozygosity (LoH) was significantly higher in embryos than couples (3733, S.D. 87 regions versus 5460, S.D. 1609). Pathogenic variations carried by either adult located within regions in embryos of allele dropout and/or low-coverage were examined manually via of loss of heterozygosity (LoH; >95% and 100 variations) for variations which were not covered by embryo coverage >10 reads. An average of 2.3 variations (SD±1.2) classified as Likely Pathogenic/Pathogenic (according to ClinVar) were missing from the embryo sequencing due to low coverage threshold or LoH, whilst all the non-PGT variations were ranked as ClinVar Review Status ‘2 star’ in terms of evidence level (0 stars representing no assertion criteria, i.e. minimal evidence, up to 4 stars for practice guideline). Missing pathogenic variations of low-coverage were phased via nearest flanking SNPs of the missing regions to determine the carrier status. A mean of 4.5 variations (S.D. 3.7) from the Likely Pathogenic or Pathogenic variations each found in embryos and 5.5 unclassified variations (S.D. 3.4) deemed potentially likely as pathogenic that required haplotype curation to account for dropout of potentially inherited but missing pathogenic variations.

To avoid filtering out true-positive de novo mutations, a ‘Goalkeeper’ filter container was used to capture clinically relevant variations for manual curation. After elimination of PGT variations, a total of 17 variations were detected in the 11 embryos with Review Status of 3 Stars, of which none were clinically actionable Essential/Developmental Delay Genes. Review Status classification revealed that only the ‘Goalkeeping’ filter container had missing calls, with a mean of 2.36 (S.D. 3.86); none of the Goalkeeping variations resulted in compound heterozygotes with inherited and transmitted variations. There were no ClinVar Review Status 1 star—‘Conflicting Interpretations’ found in any of the embryo samples. Similarly, there were no compound heterozygotes, homozygous autosomal recessive (i.e. Alt/Alt) or X-linked in females or Likely Pathogenic/Pathogenic in ACMG Incidental Findings variations in embryos or male or female partner genomes. There were 109 candidate pathogenic de novo mutations across the 11 embryos with 9 variations featured repeatedly across multiple embryos, all but two of which occurred across in more than one family. There were 14 candidate de novo autosomal dominant pathogenic variations identified (Table 3). Of these, 10 variations had a VAF <0.4, indicating with high probability most of these as false positive calls (Table 3).

TABLE 3
Autosomal dominant de novo mutations identified in the biopsied embryos.

| | | | Variation |
|---|---|---|---|
| | | Chromosome:Position; | Allele |
| Gene | Diseases | (rs#) | Frequency |
| Lamin A/C (LMNA) | Charcot-Marie-Tooth disease, | 1:156096660; | 0.3662 |
| | type 2 Muscular Dystrophy, | (rs1048086299) | |
| | Congenital, Lmna-Related | | |
| Ryanodine Receptor 2 | Ventricular Tachycardia, | 1:237942068; | 0.42105 |
| (RYR2) | Catecholaminergic | (rs754610647) | |
| | Polymorphic, 1, With Or | | |
| | Without Atrial Dysfunction | | |
| | And/Or Dilated | | |
| | Cardiomyopathy and | | |
| | Arrhythmogenic Right | | |
| | Ventricular Dysplasia, | | |
| | Familial, 2. | | |
| Zinc Finger Protein 644 | Myopia 21 | 1:91405028; | 0.38462 |
| (ZNF644) | | (rs945688793) | |
| Chromodomain | Sifrim-Hitz-Weiss Syndrome | 12:6682336 | 0.43902 |
| Helicase DNA Binding | and Cellular Schwannoma | | |
| Protein 4 (CHD4) | | | |
| Titin (TTN) | Hereditary Myopathy With | 2:179541988 | 0.35593 |
| | Early Respiratory Failure and | | |
| | Tibial Muscular Dystrophy, | | |
| | Tardive | | |
| Peroxisome | Body Mass Index Quantitative | 5:149212261; | 0.37037 |
| Proliferator-Activated | Trait Locus 11 and Kidney | (rs773389723) | |
| Receptor Gamma, | Lipoma | | |
| Coactivator 1 Beta | | | |
| (PPARGC1B) | | | |
| Succinate | Mitochondrial Complex Ii | 5:228306 | 0.31035 |
| Dehydrogenase | Deficiency | (rs775143272) | |
| Complex Flavoprotein | | | |
| Subunit A (SDHA) | | | |
| Proto-Oncogene | Congenital heart defects and | 9:133748283 | 0.62937 |
| Tyrosine-Protein | skeletal malformations | (rs121913459) | |
| Kinase/V-Abl Abelson | syndrome (CHDSKM) | | |
| Murine Leukemia Viral | | | |
| Oncogene Homolog 1 | | | |
| (ABL1) | | | |
| Collagen Type V Alpha | Ehlers-Danlos Syndrome, | 9:137703371 | 0.4 |
| 1 Chain (COL5A1) | Classic Type | | |
| Transcription Factor | Renal Cell Carcinoma, Xp11- | X:48888094 | 0.380952 |
| Binding To IGHM | Associated | | |
| Enhancer 3 (TFE3) | | | |
| Patched 1 (PTCH1) | Basal Cell Nevus Syndrome | 9:98270530 | 0.411765 |
| | and Holoprosencephaly 7 | (rs1057518664) | |
| Catechol-O- | Schizophrenia and Panic | 22:19950266 | 0.358974 |
| Methyltransferase | Disease 1 | | |
| (COMT) | | | |
| Dynein Cytoplasmic 1 | Spinal Muscular Atrophy, | 14:102452243 | 0.37931 |
| Heavy Chain 1 | Lower Extremity- | | |
| (DYNC1H1) | Predominant, 1, Autosomal | | |
| | Dominant and Charcot-Marie- | | |
| | Tooth Disease, Axonal, Type | | |
| | 2O. | | |
| Pre-B-Cell Leukemia | Congenital Anomalies Of | 1:164781366 | 0.3666 |
| Transcription Factor 1 | Kidney And Urinary Tract | | |
| (PBX1) | Syndrome With Or Without | | |
| | Hearing Loss, Abnormal Ears, | | |
| | Or Developmental Delay and | | |
| | Leukemia, Acute | | |
| | Lymphoblastic 3 | | |

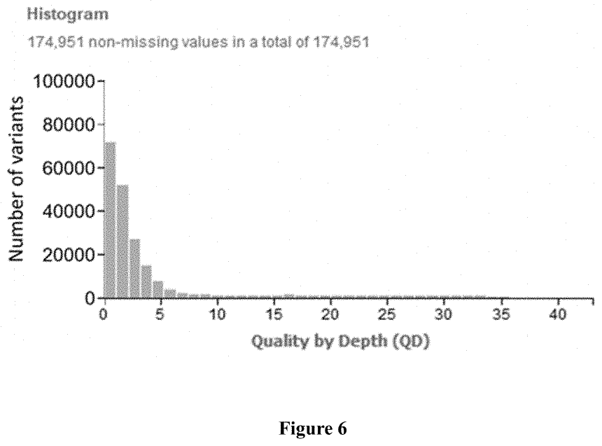

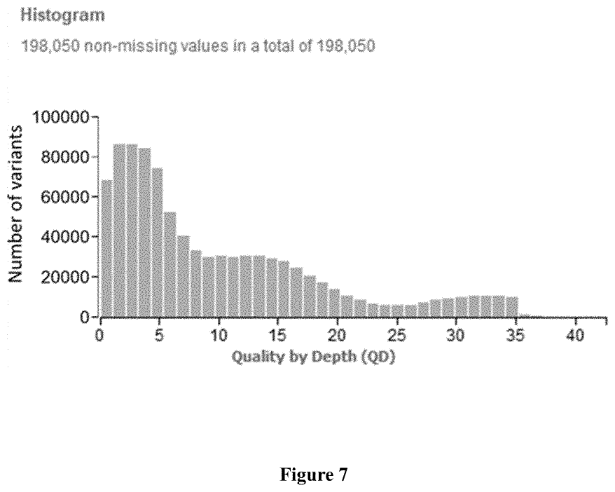

Candidate de novo mutations were also assessed according to their Quality by Depth (QD) score, as shown in FIG. 6 and FIG. 7. Due to sequencing artefacts or early-amplification phase base misincorporation, QD values in embryo de novo calls were observed to exhibit a low VAF (00.3) that coincided with QD values <12. While lower values may be used, this is one example of an appropriate false-positive filter based on QD value for identifying de novo mutations in embryos.

### Example 4—Copy-Number and Structural Variations

CNV calls were increased for amplified embryos to and parent samples with the exceptions to this being inter-chromosomal structural variations and structural deletions, suggesting a high false-positive rate (Table 1). CNVs were assessed by binning reads in 10 kb windows ClinGen Gene Dosage Sensitivity scores for pathogenicity and inheritance. As anticipated from the Karyomapping results, there were no CNVs detected which were predicted to be pathogenic. There was an average of 2.0 deleterious autosomal recessive structural variations (SVs) for both couples and embryos compared to an average of 5.21 and 8.05 SVs which triplosensitivity was contributing in couples and embryos respectively as autosomal recessive.

### Example 5—Tandem Repeat Disease Loci Analysis

For the 17 loci that Expansion Hunter assessed the tandem repeat number at known disease-causing loci, no parent samples indicated pathogenic repeat numbers. In embryo samples, the majority of the loci tested provided at least one concordant call in each embryo in terms of transmission exactness. At three loci, both alleles were discordant; FMRE ATXN1 and ATXN3.

### Discussion

The screening of the entire genomes of human embryos for pathogenic mutations was successfully accomplished, providing a proof of the feasibility of whole genome sequencing to screen biopsied IVF embryos for pathogenic genetic variations. Including introduced de novo mutations and premutation trinucleotide repeat diseases using preconception testing, the risk of childhood disease the veritable full-complement of the diseases with known aetiologies can be prevented, should any couple so choose.

As a summary, embryo biopsy samples that had undergone multiple displacement amplification (MDA) and respective parent genomic DNA samples were used as templates for generating DNA libraries which were subsequently sequenced to obtain whole genome sequencing data. Multiple trio-testing was used to classify variations as ‘Likely Pathogenic’ or ‘Pathogenic’ by disease/variation categorisation and variation filtering. Genomes of embryos and respective parents were sequenced and interpreted to detect the transmission or introduction of pathogenic mutations using databases of known pathogenic variations and functional prediction algorithms. De novo variation calling presented a unique challenge; a customised filter set minimised false-positives from MDA amplification incorporation errors from single base insertions which called at a VAF of <0.35. Determining the validity of de novo variation calls necessitated overcoming any early amplification cycle polymerase base incorporation error, allelic dropout and mis-aligned reads, producing false-positive variations. The advantages to embryo development and implantation rates conferred by the technique of trophectoderm biopsy of 4-10 cells serves as an additional benefit by maximising embryo genome sequencing coverage. Minimising amplification and sequencing artefacts through allelic ratio and haplotype scoring effectively minimises the number candidate de novo mutations to a number which can be, if necessary, manually curated.

Initial stringent filtering of de novo variations through a VAF threshold was offset by the ‘Goalkeeper’ filter set which was intended to perform the converse low-sensitivity function but which would detect clinically actionable (high risk) variations. Ultimately, the VAF threshold of 0.35 filters acted as guidance to the degree of confidence in the de novo pathogenic variations. The presence of a previously identified PGT mutation occurring with a very low VAF in one of the embryo results exemplified the need to have specific filter sets per disease/variation subtype. To circumvent the scenario of non-mendelian pathogenic variations being transmitted in low or missing coverage regions being undetected, an ‘Untransmitted’ variation filter manually examined uncalled variations flanking haplotypes to confirm the result at each site. The uniform coverage exhibited by MDA amplified DNA from embryos studied suggests the likelihood of a pathogenic de novo mutation arising in a region with low coverage is remote. These Type II error rates are mitigated by the ‘Goalkeeper’ low-coverage assessment filter, though the LoH and VAF filters can guide decision-making somewhat. Imputation was avoided for regions of LoH to focus on what could be ascertained from the data directly.

Variation detection of known ‘Likely Pathogenic’ and ‘Pathogenic’ variations was performed in accord with publicly available databases of variations with proven high to complete penetrance pathogenicity (i.e., ClinVar). Furthermore, a non-exhaustive list of genes described as Essential combined with Developmental Delay Genes was utilised to act as an additional information to provide an example of high risk variations for clinical guidance.

In conclusion, the present disclosure demonstrates the validity of utilising WGS in the IVF clinic for screening embryos for both inherited and de novo pathogenic genetic variations.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abyzov et al. (2011) Genome Research 21:974-984
Adzhubei et al. (2013) Current protocols in human genetics 7:Unit7.20-Unit7.20
Bentley et al. (2009) Nature 6:53-59
DePristo et al. (2011) Nature genetics 43:491-498
Dolzhenko et al. (2017) Genome Research
Fan et al. (2014) Curr. prot. bioinformatics 2014:10.1002/0471250953.bi1506s45
Guatelli (1990) Proc. Natl. Acad. Sci. USA 87:1874
Handyside et al. (2010) Journal of Medical Genetics 47:651-658
Harris et al. (2008) Science 320:106-109
Kearney et al. (2011) Genetics in Medicine 13:680
Kircher et al. (2014) Nature genetics 46:310-315
Kozarewa et al., (2009) Nature Methods 6:291-295
Kwoh (1989) Proc. Natl. Acad. Sci. USA 86:1173
Landegren (1988) Science 241:1077
Li et al., (2004) Clin Chem 50:1002-1011
Li and Durbin (2010) Bioinformatics 26
Margulies et al. (2005) Nature 437:376-380
McKenna et al. (2010) Genome Res 20
Metzker (2010) Nature Rev 11:31-46
Ng and Henikoff (2003) Nucleic Acids Research 31:3812-3814
Patch et al. (2018) PLoS ONE 13:e0190264
Quang et al. (2015) Bioinformatics 31; 761-763
Rehm et al. (2013) Genetics in medicine: official journal of the American College of Medical Genetics 15:733-747
Reva et al. (2011) Nucleic Acids Research 39:e118
Richards et al. (2015) Genet Med 17:405-24
Riggs et al. (2018) Human Mutation 39:1650-1659
Samocha et al. (2017) bioRxiv, 2017
Schwarz et al. (2014) Nature Methods 11:361
Shihab et al. (2014) Human Genomics 8:11
Smith (1997) J. Clin. Microbial. 35:1477-1491
Soni and Meller (2007) Clin Chem 53:1996-2001
Van der Auwera et al. (2013) Current protocols in bioinformatics 11:11.10.1-11.10.33
Volkerding et al. (2009) Clin Chem 55:641-658
Wang et al. (2011) Nature methods 8:652-654

1. A method of screening an in vitro fertilization (IVF) embryo for pathogenic genetic variations, the method comprising
a) obtaining whole genome sequencing data from the embryo, the embryo's male parent, and the embryo's female parent,
b) aligning the embryo sequencing data to a reference genome and identifying variations in the embryo sequencing data relative to the reference genome,
c) aligning the male parent's sequencing data and the female parent's sequencing data to the reference genome and identifying variations present in the parent sequencing data relative to the reference genome,
d) comparing the variations identified in step b) with those identified in step c) to identify inherited genetic variations in the embryo, wherein the inherited genetic variations are present in the embryo sequencing data and at least one parent's sequencing data,
e) filtering the variations identified in step b) that were not identified as inherited genetic variations in step d) through a variation allele frequency (VAF) threshold, wherein the filtered variations having a VAF above the threshold are identified as de novo genetic variations, and
f) comparing the inherited genetic variations and the de novo genetic variations to a database of known pathogenic genetic variations to determine if the embryo is at risk of having a pathogenic genetic variation.

2. The method of claim 1, further comprising using one or more pathogenicity prediction algorithms to predict if any of the genetic variations not identified as known pathogenic genetic variations in step f) are pathogenic genetic variations.

3. The method of claim 2, wherein the one or more pathogenicity prediction algorithms include SIFT, Polyphen2 HVAR, MutationTaster2, MutationAssessor, FATHMM, or FATHMM MKL.

4. The method of claim 3, wherein two or three or four or five or six or more pathogenicity prediction algorithms are used.

5. The method of any one of claims 1 to 4, wherein predicting a variation to be a pathogenic genetic variation requires that a variation has a MPC score of greater than about 2 and/or a Phred scaled CADD score of greater than about 20.

6. The method of any one of claims 1 to 6, wherein the VAF threshold in step e) is between 0.25 to 0.45 or 0.3 to 0.4 or is about 0.35.

7. The method of any one of claims 1 to 7, wherein the database of known pathogenic genetic variations is ClinVar.

8. The method of claim 7, wherein determining that a genetic variation is a pathogenic genetic variation in step f) requires that the genetic variation has a clinical significance value of pathogenic or likely pathogenic in the ClinVar database.

9. The method of any one of claims 1 to 8, wherein the genetic variations include single nucleotide polymorphisms (SNPs), insertions or deletions (indels), copy number variations (CNVs), and/or structural variations.

10. The method of any one of claims 1 to 9, wherein the inherited genetic variations include autosomal dominant, autosomal recessive, compound heterozygous, and/or X-linked genetic variations.

11. The method of any one of claims 1 to 10, wherein the whole genome sequencing data from the embryo is obtained by
a) culturing the embryo,
b) performing a biopsy on the embryo,
c) amplifying genomic DNA from the biopsy, and
d) sequencing the amplified genomic DNA.

12. The method of claim 11, wherein the biopsy is a trophectoderm biopsy.

13. The method of claim 12, wherein the trophectoderm biopsy is performed on day 5 or day 6 of culture.

14. The method of any one of claims 11 to 13, wherein the genomic DNA is amplified from the biopsy using multi-displacement amplification (MDA).

15. The method of any one of claims 12 to 14, wherein the genomic DNA is sequenced using DNA nanoball sequencing.

16. The method of claim 15, wherein the DNA nanoball sequencing is performed with combinatorial probe anchor ligation (cPAL).

17. The method of any one of claims 1 to 16, wherein the reference genome is a Genome Reference Consortium Human Build.

18. The method of any one of claims 1 to 17, wherein two or more embryos from the same parents are screened.

19. The method of any one of claims 1 to 18, further comprising transferring the embryo into the female parent's, or a surrogate's, uterus.

20. The method of any one of claims 1 to 19, wherein the embryo is a human embryo.

21. An IVF process comprising
a) fertilizing an egg from a female parent with a sperm from a male parent,
b) culturing the fertilized egg, thereby producing an embryo,
c) screening the embryo for pathogenic genetic variations using the method of any one of claims 1 to 20, and
d) transferring the embryo into the female parent's, or a surrogate's, uterus.

22. A method of screening an in vitro fertilization (IVF) embryo for one or more phenotypic traits, the method comprising
a) obtaining whole genome sequencing data from the embryo, the embryo's male parent, and the embryo's female parent,
b) aligning the embryo sequencing data to a reference genome and identifying variations in the embryo sequencing data relative to the reference genome,
c) aligning the male parent's sequencing data and the female parent's sequencing data to the reference genome and identifying variations present in the parent sequencing data relative to the reference genome,
d) comparing the variations identified in step b) with those identified in step c) to identify inherited genetic variations in the embryo, wherein the inherited genetic variations are present in the embryo sequencing data and at least one parent's sequencing data,
e) filtering the variations identified in step b) that were not identified as inherited genetic variations in step d) through a variation allele frequency (VAF) threshold, wherein the filtered variations having a VAF above the threshold are identified as de novo genetic variations, and
f) comparing the inherited genetic variations and the de novo genetic variations to a database of genetic variations having known phenotypic traits to determine if the embryo has the one or more phenotypic traits.

23. The steps, features, integers, compositions and/or compounds disclosed herein or indicated in the specification of this application individually or collectively, and any and all combinations of two or more of said steps or features.
